(19) United States
(12) Patent Application Publication
(10) Pub. No.: US 2023/0226313 A1
(43) Pub. Date: Jul. 20, 2023

(54) Vascular Access System with Secondary Catheter

(71) Applicant: Becton, Dickinson and Company, Franklin Lakes, NJ (US)

(72) Inventors: Jonathan Karl Burkholz, Salt Lake City, UT (US); Megan S. Scherich, Salt Lake City, UT (US)

(21) Appl. No.: 18/092,583

(22) Filed: Jan. 3, 2023

Related U.S. Application Data
(60) Provisional application No. 63/296,302, filed on Jan. 4, 2022.

(51) Int. Cl. A61M 25/01; A61M 39/02

(52) U.S. Cl. CPC A61M 25/01; A61M 39/02; A61M 2039/062

(57) ABSTRACT

A vascular access system includes a catheter adapter having a body and a primary catheter configured to be inserted into a patient’s vasculature, with the body having a first end and a second end positioned opposite the first end, a primary lumen in fluid communication with the primary catheter, a primary lumen port in fluid communication with the primary lumen, a secondary lumen in fluid communication with the primary catheter, a secondary lumen port in fluid communication with the secondary lumen, and a mid-line lumen at least partially received within the secondary lumen. The mid-line lumen having a retracted position where a distal end of the mid-line lumen is positioned within at least one of the primary catheter and the secondary lumen, and an extended position where the distal end of the mid-line lumen extends beyond the distal end of the primary catheter.

### CROSS-REFERENCE TO RELATED APPLICATION

The present application claims priority to U.S. Provisional Application No. 63/296,302, entitled “Vascular Access System with Secondary Catheter”, filed Jan. 4, 2022, the entire disclosure of which is hereby incorporated by reference.

### BACKGROUND OF THE INVENTION

### Field of the Invention

The present disclosure relates to a vascular access system with a secondary catheter.

### Description of Related Art

Catheters are frequently utilized to administer fluids into and out of the body. Patients in a variety of settings, including in hospitals and in home care, receive fluids, pharmaceuticals, and blood products via a vascular access device inserted into a patient’s vascular system. Catheters of various types and sizes have been used extensively in a variety of procedures including, but not limited to, treating an infection, providing anesthesia or analgesia, providing nutritional support, treating cancerous growths, maintaining blood pressure and heart rhythm, and many other clinical uses. A common vascular access device is a plastic catheter that is inserted into a patient’s vein. The catheter length may vary from a few centimeters for peripheral access to many centimeters for central access. The catheter is commonly incorporated into a catheter adapter to aid in the ease of use, accessibility and utility of the catheter. A catheter adapter may be adapted to house one end of the catheter such that one end of the catheter is supported by the catheter adapter and the body and tip of the catheter extends beyond a first end of the catheter adapter. A catheter adapter generally further includes a second end adapted to receive additional infusion components for use with the catheter. For example, the second end of a catheter adapter may include a set of threads for attaching an intravenous line or for coupling a syringe to the catheter adapter thereby providing access to the patient’s vasculature via the attached catheter.

The catheter may be inserted transcutaneously. When inserted transcutaneously, the insertion of the catheter is commonly aided by an introducer needle. The introducer needle is commonly housed inside the lumen of the catheter such that the gauge of the needle approximates the inner diameter of the catheter. The needle is positioned within the catheter such that the needle tip extends beyond the tip of the catheter whereby the needle is used to penetrate the patient’s vein and provide an opening for insertion of the catheter.

In order to verify proper placement of the introducer needle and/or the catheter in the blood vessel, a clinician generally confirms that there is “flashback” of blood in a flashback chamber of the catheter assembly. Once placement of the needle has been confirmed, the clinician may temporarily occlude flow in the vasculature and remove the needle, leaving the catheter in place for future blood withdrawal, fluid infusion, or probe access.

Blood withdrawal or infusion using the catheter may be difficult for several reasons, particularly when a dwelling time of the catheter within the patient is more than one day. For example, when the catheter is left inserted in the patient for a prolonged period of time, the vein and catheter may be more susceptible to narrowing, collapse, kinking, blockage by debris (e.g., fibrin, platelet clots, or thrombus), and adhering of a tip of the catheter to the vasculature. Due to this, catheters may often be used for acquiring a blood sample at a time of catheter placement but are much less frequently used for acquiring a blood sample during the catheter dwell period. Therefore, when a blood sample is desired, an additional needle stick is used to provide vein access for blood collection, which may be painful for the patient and result in higher material costs.

### SUMMARY OF THE INVENTION

In one aspect or embodiment, a vascular access system includes a catheter adapter having a body and a primary catheter configured to be inserted into a patient’s vasculature, with the body having a first end and a second end positioned opposite the first end, a primary lumen in fluid communication with the primary catheter, a primary lumen port in fluid communication with the primary lumen, a secondary lumen in fluid communication with the primary catheter, a secondary lumen port in fluid communication with the secondary lumen, and a mid-line lumen at least partially received within the secondary lumen, with the mid-line lumen having a retracted position where a distal end of the mid-line lumen is positioned within at least one of the primary catheter and the secondary lumen, and an extended position where the distal end of the mid-line lumen extends beyond a distal end of the primary catheter.

The vascular access system may include an advancement member configured to be grasped by a healthcare technician, where movement of the advancement member moves the mid-line lumen between the retracted position and the extended position. The advancement member may be configured to move along an outer surface of the secondary lumen, where the advancement member is entirely positioned outside of the secondary lumen.

The body of the catheter adapter may include a side port positioned between the first end and the second end of the body, where the primary lumen and the secondary lumen are in fluid communication via a joining connector. The joining connector may be in fluid communication with the side port via intermediate tubing. The joining connector may be formed integrally with the body of the catheter adapter. The primary lumen and the secondary lumen may be permanently joined to the joining connector. The primary lumen may be permanently joined to the joining connector, where the secondary lumen and the mid-line lumen are joined to the joining connector via a connector. The joining connector may be a needle-free connector. A longitudinal axis of the secondary lumen may be configured to be aligned with a longitudinal axis of the intermediate tubing and a longitudinal axis of the side port of the body of the catheter adapter.

The primary lumen port may be a first needle-free connector at a proximal end of the primary lumen and the secondary lumen port may be a second needle-free connector at a proximal end of the secondary lumen. The vascular access system may include a blood collection device configured to be connected to and in fluid communication with the secondary lumen.

The primary lumen and the secondary lumen may be in fluid communication via a joining connector, where the joining connector is attached to the second end of the body of the catheter adapter via a connector. A longitudinal axis of the secondary lumen may be configured to be aligned with a longitudinal axis of the primary catheter.

A flow path through the secondary lumen to the primary catheter may be open when the mid-line lumen is in the retracted position and closed when the mid-line lumen is in the extended position. The primary lumen and the secondary lumen may be in fluid communication via a joining connector, where the joining connector includes a seal configured to close the flow path through the secondary lumen to the primary catheter when the mid-line lumen is in the extended position.

The mid-line lumen may include a first portion extending from the distal end of the mid-line lumen to a position intermediate the distal end of the mid-line lumen and a proximal end of the mid-line lumen and a second portion extending from the proximal end of the mid-line lumen to the first portion of the mid-line lumen, where the first portion includes a first material and the second portion includes a second material, and where the second material is stiffer than the first material.

The mid-line lumen may include a tapered portion, where the tapered portion of the mid-line lumen engages the seal when the mid-line lumen is in the extended position.

The primary lumen and the secondary lumen may be in fluid communication via a joining connector, where the mid-line lumen includes a seal configured to engage a portion of the joining connector to close the flow path through the secondary lumen to the primary catheter when the mid-line lumen is in the extended position.

The primary lumen and the secondary lumen may be in fluid communication via a joining connector, where a seal actuator is at least partially received within the joining connector, and where movement of the seal actuator is configured to close a flow path through the secondary lumen to the primary catheter.

The distal end of the mid-line lumen may include a slit having an open position allowing fluid to flow out of or into the mid-line lumen and a closed position. A distal end of the primary catheter may include an opening defined by a sidewall of the primary catheter.

In one aspect or embodiment, a method of using the vascular access system includes: attaching a blood collection device to the secondary lumen port; moving the mid-line lumen to the extended position; and removing the blood collection device from the secondary lumen port.

In one aspect or embodiment, a method of using the vascular access system includes attaching a fluid source to the secondary lumen port; moving the mid-line lumen to the extended position; and delivering fluid from the fluid source through the mid-line lumen.

### BRIEF DESCRIPTION OF THE DRAWINGS

The above-mentioned and other features and advantages of this disclosure, and the manner of attaining them, will become more apparent and the disclosure itself will be better understood by reference to the following descriptions of embodiments of the disclosure taken in conjunction with the accompanying drawings, wherein:

FIG. 1 is a perspective view of a vascular access system according to one aspect or embodiment of the present application;

FIG. 2 is a perspective view of a vascular access system according to a further aspect or embodiment of the present application, showing a retracted positon of a mid-line lumen;

FIG. 3 is a perspective view of the vascular access system of FIG. 2, showing an extended position of a mid-line lumen;

FIG. 4 is a perspective view of the vascular access system of FIG. 2, showing a secondary lumen connected to a blood collection device;

FIG. 5 is a perspective view of the vascular access system of FIG. 2, showing a secondary lumen connected to a blood collection device;

FIG. 6 is a perspective view of a vascular access system according to a further aspect or embodiment of the present application, showing an extended positon of a mid-line lumen;

FIG. 7 is a perspective view of a primary catheter and mid-line lumen according to one aspect or embodiment of the present application, showing the primary catheter and the mid-line lumen inserted into the vasculature of a patient;

FIG. 8 is a schematic view of a vascular access system according to a further aspect or embodiment of the present application;

FIG. 9A is a schematic view of a vascular access system according to a further aspect or embodiment of the present application, showing a retracted position of a mid-line lumen;

FIG. 9B is a schematic view of the vascular access system of FIG. 9A, showing an extended position of a mid-line lumen;

FIG. 10A is a perspective view of a vascular access system according to a further aspect or embodiment of the present application, showing a retracted positon of a mid-line lumen;

FIG. 10B is a perspective view of the vascular access system of FIG. 10A, showing a retracted positon of a mid-line lumen;

FIG. 11 is a perspective view of a vascular access system according to a further aspect or embodiment of the present application, showing a retracted positon of a mid-line lumen;

FIG. 12 is a perspective view of the vascular access system of FIG. 11, showing an extended positon of a mid-line lumen;

FIG. 13 is a perspective view of the vascular access system of FIG. 11, showing an extended positon of a mid-line lumen with a blood collection device connected to a secondary lumen;

FIG. 14 is a perspective view of a vascular access system according to a further aspect or embodiment of the present application;

FIG. 15 is a perspective view of a vascular access system according to a further aspect or embodiment of the present application, showing an extended positon of a mid-line lumen with a blood collection device connected to a secondary lumen;

FIG. 16 is a partial cross-sectional view of a secondary lumen and an advancement member according to one aspect or embodiment of the present application;

FIG. 17 is a schematic view of a vascular access system according to a further aspect or embodiment of the present application;

FIG. 18 is a schematic view of a vascular access system according to a further aspect or embodiment of the present application;

FIG. 19 is a schematic view of a vascular access system according to a further aspect or embodiment of the present application;

FIG. 20 is a schematic view of a vascular access system according to a further aspect or embodiment of the present application;

FIG. 21 is a schematic view of a primary catheter and mid-line lumen according to one aspect or embodiment of the present application;

FIG. 22 is a schematic view of the primary catheter and mid-line lumen of FIG. 21, showing a deflected position of the mid-line lumen;

FIG. 23 is a schematic view of a mid-line lumen according to one aspect or embodiment of the present application;

FIG. 24 is a schematic view of a mid-line lumen according to one aspect or embodiment of the present application;

FIG. 25 is a schematic view of a mid-line lumen according to one aspect or embodiment of the present application;

FIG. 26 is a schematic view of a mid-line lumen according to one aspect or embodiment of the present application; and

FIG. 27 is a schematic view of a mid-line lumen according to one aspect or embodiment of the present application.

Corresponding reference characters indicate corresponding parts throughout the several views. The exemplifications set out herein illustrate exemplary embodiments of the disclosure, and such exemplifications are not to be construed as limiting the scope of the disclosure in any manner.

### DETAILED DESCRIPTION OF THE INVENTION

Spatial or directional terms, such as “left”, “right”, “inner”, “outer”, “above”, “below”, and the like, are not to be considered as limiting as the invention can assume various alternative orientations.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary aspects of the invention.

Unless otherwise indicated, all ranges or ratios disclosed herein are to be understood to encompass the beginning and ending values and any and all subranges or subratios subsumed therein. For example, a stated range or ratio of “1 to 10” should be considered to include any and all subranges or subratios between (and inclusive of) the minimum value of 1 and the maximum value of 10; that is, all subranges or subratios beginning with a minimum value of 1 or more and ending with a maximum value of 10 or less.

The terms “first”, “second”, and the like are not intended to refer to any particular order or chronology, but refer to different conditions, properties, or elements.

As used herein, “at least one of” is synonymous with “one or more of”. For example, the phrase “at least one of A, B, and C” means any one of A, B, or C, or any combination of any two or more of A, B, or C. For example, “at least one of A, B, and C” includes one or more of A alone; or one or more of B alone; or one or more of C alone; or one or more of A and one or more of B; or one or more of A and one or more of C; or one or more of B and one or more of C; or one or more of all of A, B, and C.

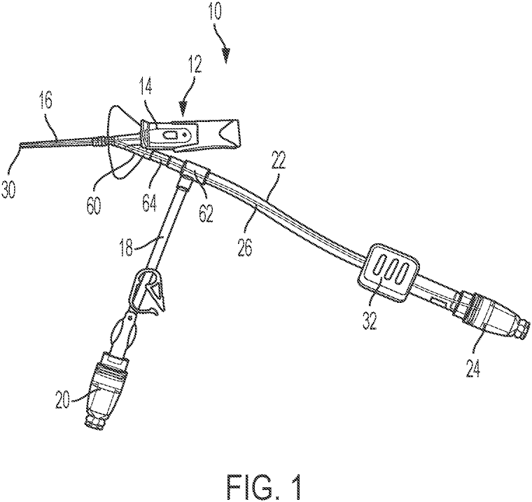

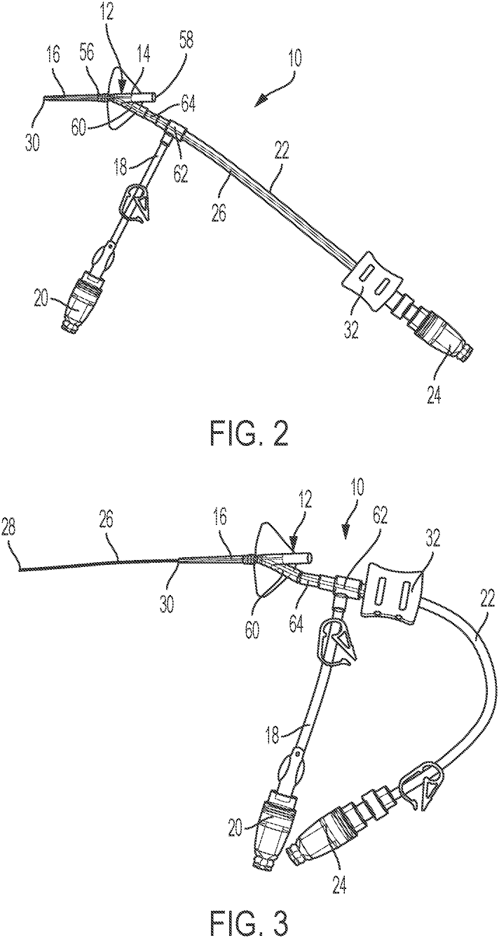

Referring to FIGS. 1-5, a vascular access system 10 according to one aspect or embodiment of the present application includes a catheter adapter 12 having a body 14 and a primary catheter 16 configured to be inserted into a patient’s vasculature, a primary lumen 18 in fluid communication with the primary catheter 16, a primary lumen port 20 in fluid communication with the primary lumen 18, a secondary lumen 22 in fluid communication with the primary catheter 16, a secondary lumen port 24 in fluid communication with the secondary lumen 22, and a mid-line lumen 26 at least partially received within the secondary lumen 22. The mid-line lumen 26 has a retracted position (FIG. 2) where a distal end 28 of the mid-line lumen 26 is positioned within at least one of the primary catheter 16 and the secondary lumen 22, and an extended position (FIG. 3) where the distal end 28 of the mid-line lumen 26 extends beyond a distal end 30 of the primary catheter 16. The vascular access system 10 is configured to extend the reach of the primary catheter 16 into a patient’s vascular system for short term needs, such as collecting a blood sample, as well as longer-term needs, such as providing multi-lumen mid-line catheter capability. The vascular access system 10 is configured to utilize the mid-line lumen 26 for blood draw and administering medication while also providing the option of simultaneously infusing fluids through the primary lumen 18.

Referring to FIGS. 2, 3, and 16, in one aspect or embodiment, the vascular access system 10 includes an advancement member 32 configured to be grasped by a healthcare technician. Movement of the advancement member 32 moves the mid-line lumen 26 between the retracted position (FIG. 2) and the extended position (FIG. 3). The advancement member 32 is configured to move along an outer surface of the secondary lumen 22, with the advancement member 32 entirely positioned outside of the secondary lumen 22. As shown in FIG. 16, the advancement member 32 includes a body 34 having gripping surfaces 36 and ball bearings 38 that engage the outer surface of the secondary lumen 22. In one aspect or embodiment, rather than providing the ball bearings 38, the body 34 of the advancement member 32 may include bumps, such as cylindrical or spherical bumps, that engage the outer surface of the secondary lumen 22. A coupler 40 is positioned within the secondary lumen 22, with the mid-line lumen 22 attached to the coupler 40. The coupler 40 is positioned between the ball bearings 38. Movement of the advancement member 32 distally or proximally causes the ball bearings 38 to engage the coupler, with the secondary lumen depressed and positioned between the ball bearings and the coupler 40, thereby moving the coupler 40 and the mid-line lumen 26 along with the advancement member 32. The advancement member 32 may be the same or similar to the translation handle shown and described in U.S. Pat. Application Publication No. 2021/0290126, which is hereby incorporated by reference in its entirety.

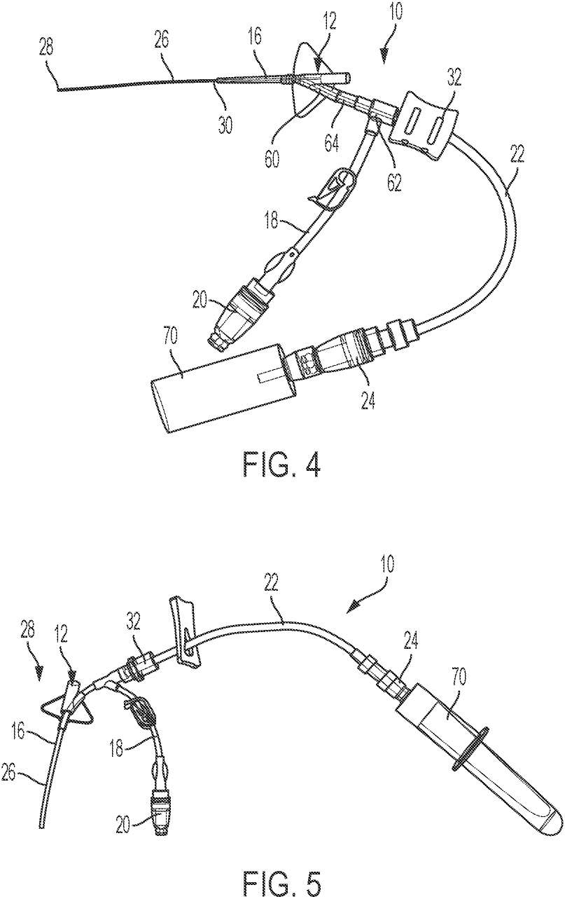

Referring again to FIGS. 1-5, the body 14 of the catheter adapter 12 has a first end 56 and a second end 58 positioned opposite the first end 56. The body 14 includes a side port 60 positioned between the first end 56 and the second end 58 of the body 14, with the primary lumen 18 and the secondary lumen 22 in fluid communication via a joining connector 62. The joining connector 62 is in fluid communication with the side port 60 via intermediate tubing 64. The primary lumen 18 and the secondary lumen 22 are permanently joined to the joining connector 62, such as through bonding, adhesive, or other suitable arrangement. A longitudinal axis of the secondary lumen 22 is configured to be aligned with a longitudinal axis of the intermediate tubing 64 and a longitudinal axis of the side port 60 of the body 14 of the catheter adapter 12. Optionally, the intermediate tubing port may join the catheter adapter body at a location offset from the primary catheter adapter body access. This offset location may be either below or above the primary catheter adapter body access to create a vortex action when fluid is delivered, and/or to improve flushing the catheter adapter to clear our any residual blood or medication. Accordingly, the secondary lumen 22, the joining connector 62, the intermediate tubing 64, the side port 60, and the primary catheter 16 define a non-tortuous path to facilitate the passage of the mid-line lumen 26 through the primary catheter 16 and into the vascular system of a patient. In one aspect or embodiment, the primary lumen port 20 is a first needle-free connector at a proximal end of the primary lumen 18 and the secondary lumen port 24 is a second needle-free connector at a proximal end of the secondary lumen 22. As shown in FIGS. 4 and 5, in certain aspects or embodiments, the vascular access system 10 includes a blood collection device 70 configured to be connected to and in fluid communication with the secondary lumen 22. The blood collection device 70, such as the BD Vacutainer® Luer-Lok™ access device, may be connected to the secondary lumen port 24.

The vascular access system 10 of FIGS. 1-5 is configured to function similarly to a standard integrated peripheral intravenous catheter with the ability to infuse through the primary lumen port 20 and the secondary lumen port 24. When flushing through the secondary lumen port 24, fluid flows through the secondary lumen 22 and through the mid-line lumen 26 to ensure the respective lumens remain flushed. When the mid-line lumen 26 is in the extended position, the mid-line lumen 26 can be utilized for delivering caustic medication. Delivering medications further downstream in a patient’s vein away from the distal end 30 of the primary catheter 16 allows for quicker hemodilution and reduces the risk of chemical phlebitis at the location of the primary catheter 16.

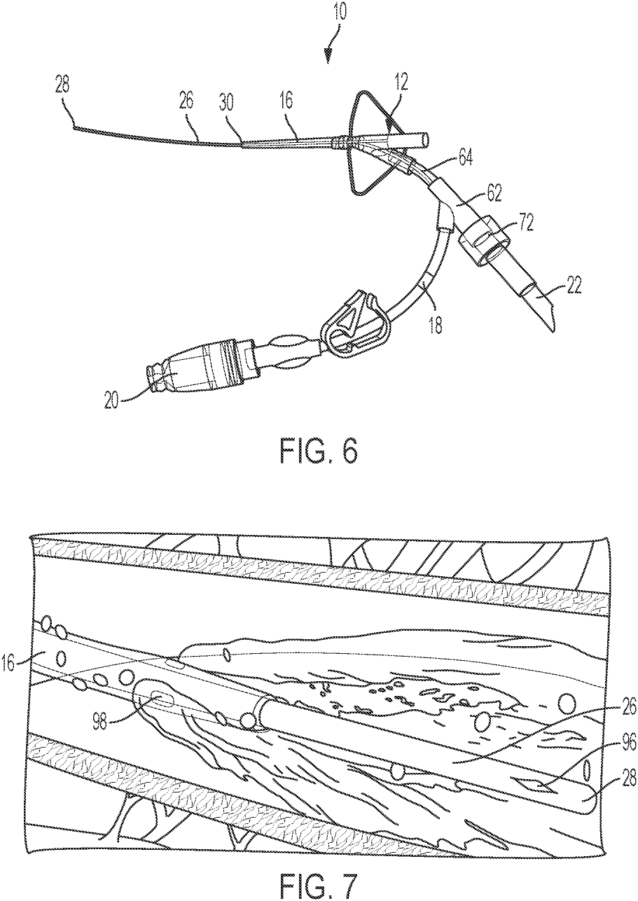

Referring to FIG. 6, in one aspect or embodiment, the secondary lumen 22 is joined to the joining connector via a connector 72, such as a luer connector or needle-free connector.

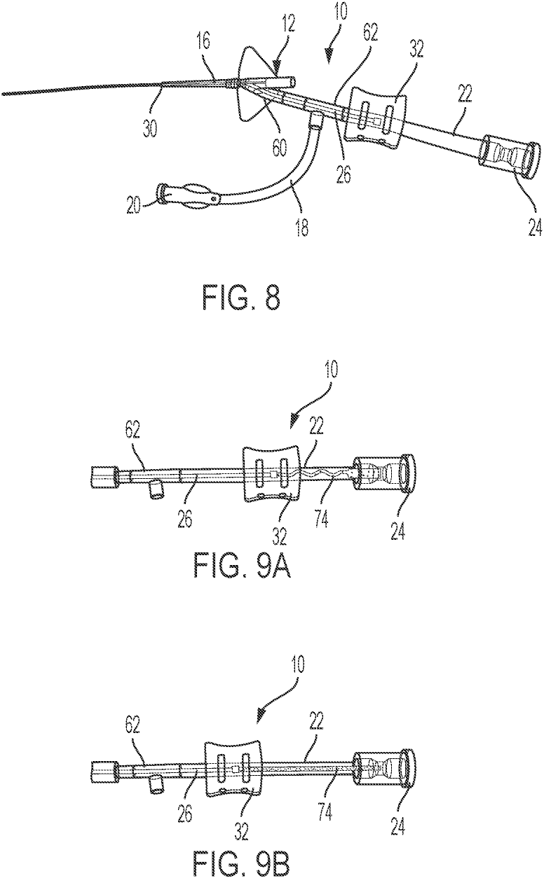

Referring to FIG. 8, in one aspect or embodiment, the joining connector 62 is formed integrally with the body 14 of the catheter adapter 12. The joining connector 62 may be molded together with the catheter adapter 12 or may be formed separately and joined or bonded to the catheter adapter 12.

Referring to FIGS. 9A and 9B, in one aspect or embodiment, the mid-line lumen 26 includes a collapsible portion 74 that is configured to be collapsed when the mid-line lumen 26 is in the retracted position and straight or straightened when the mid-line lumen 26 is in the extended position.

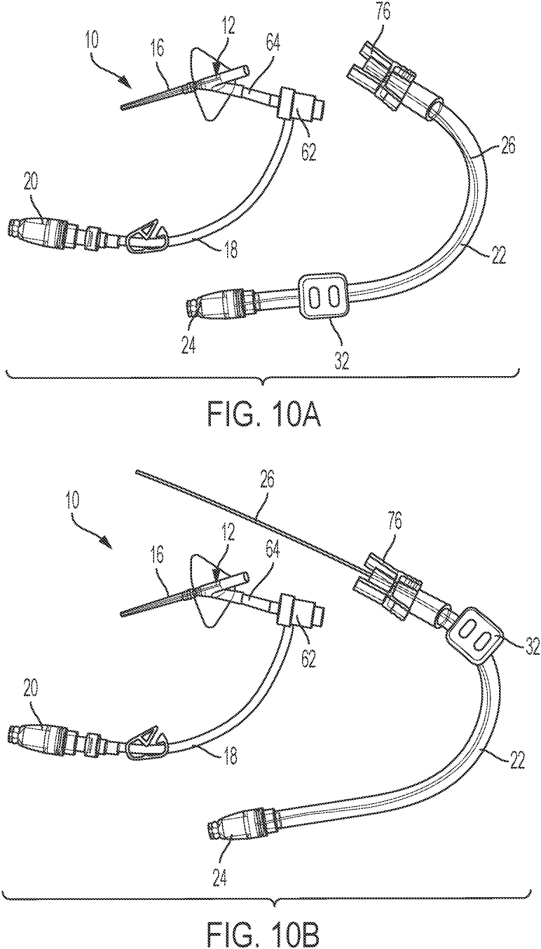

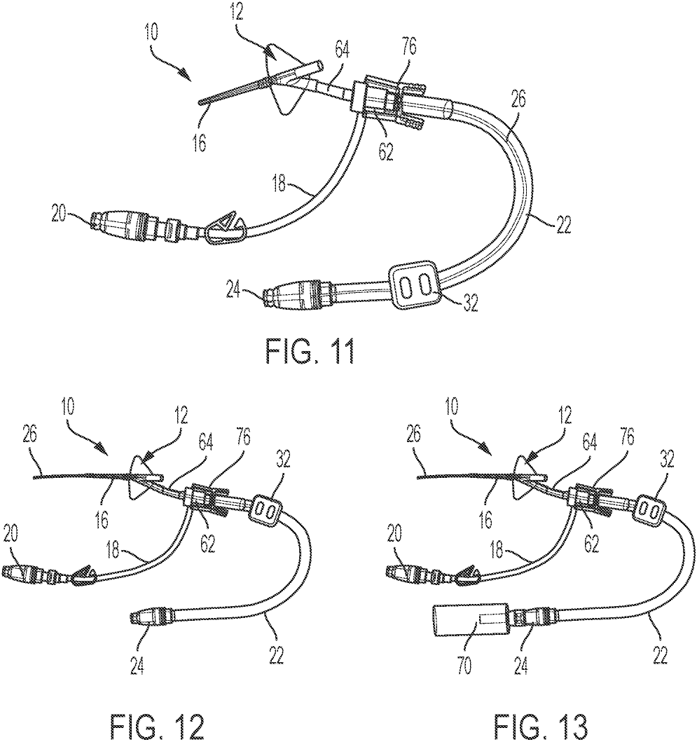

Referring to FIGS. 10A-13, in one aspect or embodiment, the primary lumen 18 is permanently joined to the joining connector 62, and the secondary lumen 22 and the mid-line lumen 26 are joined to the joining connector 62 via a connector 76. The joining connector 62 may be a needle-free connector, although other suitable connectors may be utilized. The mid-line lumen 26 is shown in the retracted position (FIG. 11), the extended position for delivering medication (FIG. 12), and the extended position for drawing blood (FIG. 13).

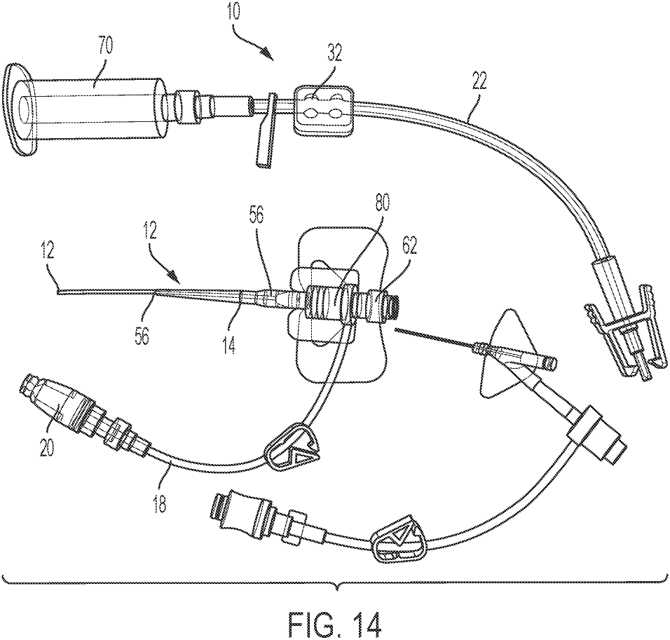

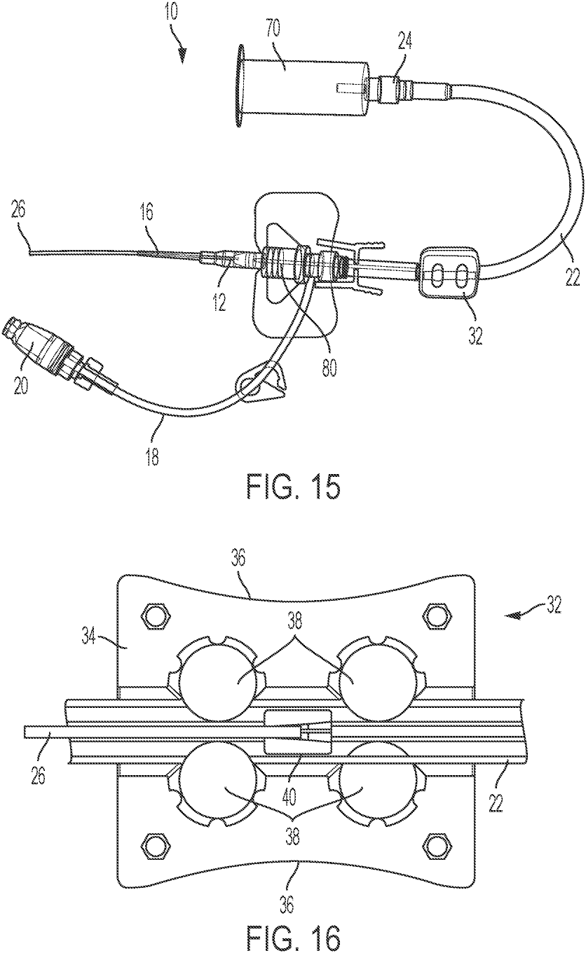

Referring to FIGS. 14 and 15, in one aspect or embodiment, the primary lumen 18 and the secondary lumen 22 are in fluid communication via the joining connector 62, with the joining connector 62 attached to the second end 58 of the body 14 of the catheter adapter 12 via a connector 80, such as a luer connector, although other suitable connection arrangements may be utilized. A longitudinal axis of the secondary lumen 22 is configured to be aligned with a longitudinal axis of the primary catheter 16, which provides a non-tortuous path for the mid-line lumen 26.

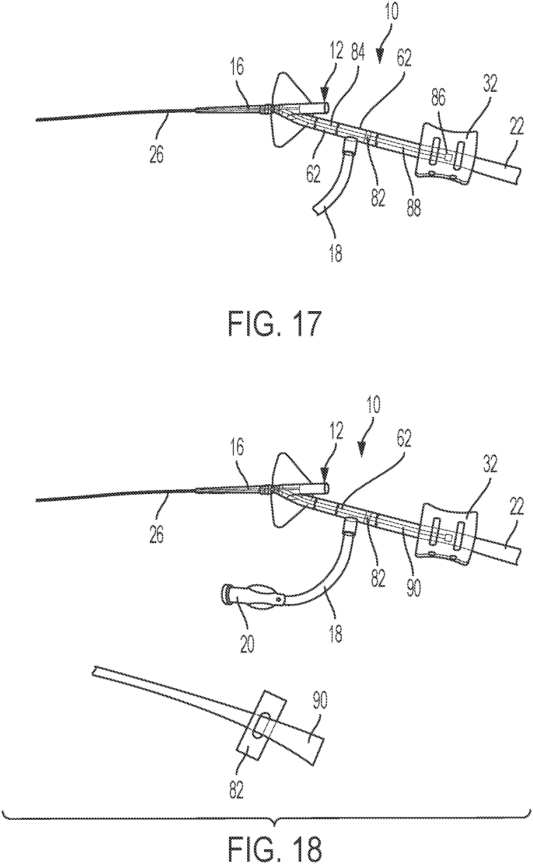

Referring to FIGS. 17-20, in one aspect or embodiment, a flow path through the secondary lumen 22 to the primary catheter 16 is open when the mid-line lumen 26 is in the retracted position and closed when the mid-line lumen 26 is in the extended position. The joining connector 62 may include a seal 82 configured to close the flow path through the secondary lumen 22 to the primary catheter 16 when the mid-line lumen 26 is in the extended position.

Referring to FIG. 17, in one aspect or embodiment, the mid-line lumen 26 includes a first portion 84 extending from the distal end 28 of the mid-line lumen 26 to a position intermediate the distal end 28 of the mid-line lumen 26 and a proximal end 86 of the mid-line lumen 26 and a second portion 88 extending from the proximal end 86 of the mid-line lumen 26 to the first portion 84 of the mid-line lumen 26. The first portion 84 is formed from a first material and the second portion 88 is formed from a second material, where the second material is stiffer than the first material. The stiffer second material can push through the seal 82 within the joining connector 62. The first portion 84 of the mid-line lumen 26 may be bonded to the second portion 88 of the mid-line lumen 26 or may be connected to each other via an interference fit.

Referring to FIG. 18, in one aspect or embodiment, the mid-line lumen 26 includes a tapered portion 90, where the tapered portion 90 of the mid-line lumen 26 engages the seal 82 within the joining connector 62 when the mid-line lumen 26 is in the extended position.

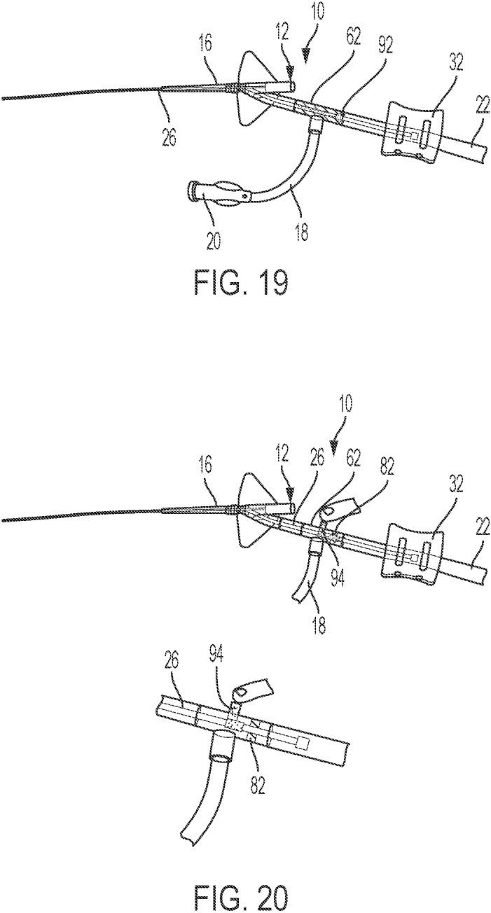

Referring to FIG. 19, in one aspect or embodiment, the mid-line lumen 26 includes a seal 92 configured to engage a portion of the joining connector 62 to close the flow path through the secondary lumen 22 to the primary catheter 16 when the mid-line lumen 26 is in the extended position. More specifically, the seal 92 is secured to an outer surface of the mid-line lumen 26 and positioned within the secondary lumen 22. When the mid-line lumen 26 is advanced to the extended position, the seal 92 engages a portion of the joining connector 62 to close the secondary lumen 22. In one aspect or embodiment, the seal 92 is tapered and engages a tapered portion of the joining connector 62.

Referring to FIG. 20, in one aspect or embodiment, the vascular access system 10 includes a seal actuator 94 at least partially received within the joining connector 62. Movement of the seal actuator 94 is configured to close a flow path through the secondary lumen 22 to the primary catheter 16. A healthcare technician can move the seal actuator 94 to open or close the flow path through the secondary lumen 22, as desired. The seal actuator 94 may be hollow to allow the mid-line lumen 26 to pass through the seal actuator 94.

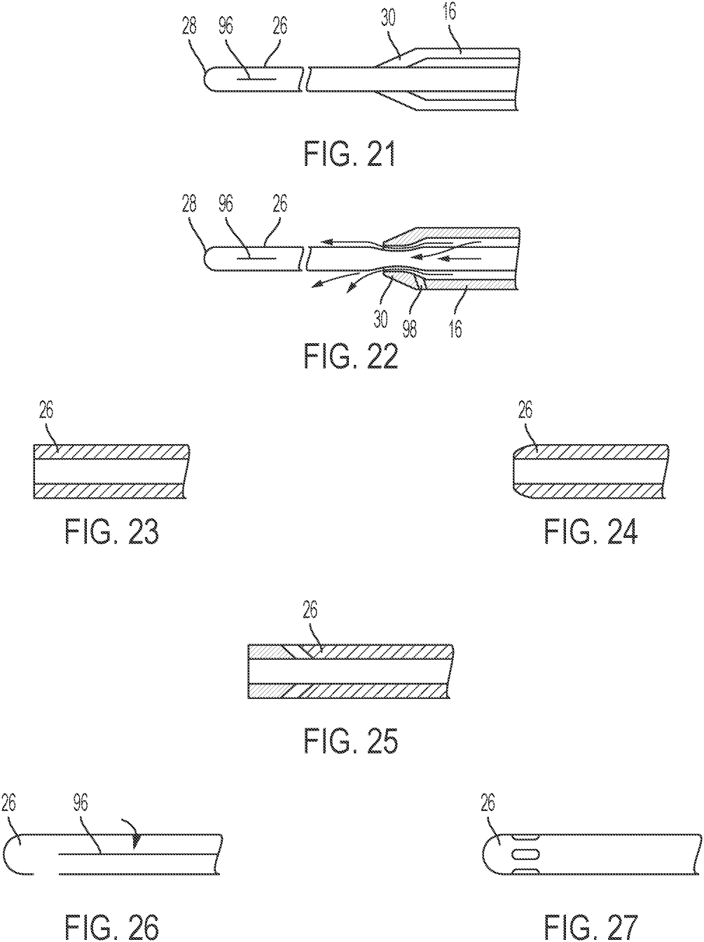

Referring to FIGS. 7, 21, and 22, in one aspect or embodiment, the distal end 28 of the mid-line lumen 26 is closed and defines a slit 96 having an open position allowing fluid to flow out of or into the mid-line lumen 26 and into a closed position. The distal end 30 of the primary catheter 16 may include an opening 98 defined by a sidewall of the primary catheter 16 to allow for the passage of fluids between the primary catheter 16 and the mid-line lumen 26.

Referring to FIGS. 23-27, the distal end 28 of the mid-line lumen 26 may include various shapes and configurations, including a standard, open-ended tip (FIG. 23), a shaped tip (FIG. 24), a diffuser tip (FIG. 25), a valve tip (FIG. 26), and a blunt tip with openings (FIG. 27), although other suitable configurations may be utilized with the vascular access system 10. The mid-line lumen 26 may be formed from a flexible material that is more gentle on the vein and able to move and flex with the fluid during the flush advancement. The material of the mid-line lumen 26 may be rigid, semi-rigid, or a flexible polymeric material, such as polyurethane, polyimide, silicon, TPE, TPU, or other polymer and/or elastomeric material. An outer diameter of the mid-line lumen 26 may vary along the length of the mid-line lumen 26 to maximize flow rate, provide for sealing features, and/or optimize indwell time. A lubricant may be added to the mid-line lumen 26 to facilitate advancement of the mid-line lumen 26. A lubricant may also be added to the primary lumen 18, the secondary lumen 22, the ball bearings 28, and/or other components of the vascular access system 10. The secondary lumen 22 may have markings to indicate how far advanced the mid-line lumen 26 is advanced relative to other portions of the vascular access system 10.

In one aspect or embodiment, a method of using the vascular access system 10 includes: attaching the blood collection device 70 to the secondary lumen port 24; moving the mid-line lumen 26 to the extended position; and removing the blood collection device 70 from the secondary lumen port 24. The method may also include delivering medication through the mid-line lumen.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

The invention claimed is:

1. A vascular access system comprising:
a catheter adapter comprising a body and a primary catheter configured to be inserted into a patient’s vasculature, the body having a first end and a second end positioned opposite the first end;
a primary lumen in fluid communication with the primary catheter;
a primary lumen port in fluid communication with the primary lumen;
a secondary lumen in fluid communication with the primary catheter;
a secondary lumen port in fluid communication with the secondary lumen; and
a mid-line lumen at least partially received within the secondary lumen, the mid-line lumen having a retracted position where a distal end of the mid-line lumen is positioned within at least one of the primary catheter, the catheter adapter, and the secondary lumen, and an extended position where the distal end of the mid-line lumen extends beyond a distal end of the primary catheter.

2. The vascular access system of claim 1, further comprising an advancement member configured to be grasped by a healthcare technician, wherein movement of the advancement member moves the mid-line lumen between the retracted position and the extended position.

3. The vascular access system of claim 2, wherein the advancement member is configured to move along an outer surface of the secondary lumen, and wherein the advancement member is entirely positioned outside of the secondary lumen.

4. The vascular access system of claim 1, wherein the body of the catheter adapter comprises a side port positioned between the first end and the second end of the body, and wherein the primary lumen and the secondary lumen are in fluid communication via a joining connector.

5. The vascular access system of claim 4, wherein the joining connector is in fluid communication with the side port via intermediate tubing.

6. The vascular access system of claim 4, wherein the joining connector is formed integrally with the body of the catheter adapter.

7. The vascular access system of claim 4, wherein the primary lumen and the secondary lumen are permanently joined to the joining connector.

8. The vascular access system of claim 4, wherein the primary lumen is permanently joined to the joining connector, and wherein the secondary lumen and the mid-line lumen are joined to the joining connector via a connector.

9. The vascular access system of claim 8, wherein the joining connector comprises a needle-free connector.

10. The vascular access system of claim 5, wherein a longitudinal axis of the secondary lumen is configured to be aligned with a longitudinal axis of the intermediate tubing and a longitudinal axis of the side port of the body of the catheter adapter.

11. The vascular access system of claim 1, wherein the primary lumen port comprises a first needle-free connector at a proximal end of the primary lumen and the secondary lumen port comprises a second needle-free connector at a proximal end of the secondary lumen.

12. The vascular access system of claim 1, further comprising a blood collection device configured to be connected to and in fluid communication with the secondary lumen.

13. The vascular access system of claim 1, wherein the primary lumen and the secondary lumen are in fluid communication via a joining connector, and wherein the joining connector is attached to the second end of the body of the catheter adapter via a connector.

14. The vascular access system of claim 13, wherein a longitudinal axis of the secondary lumen is configured to be aligned with a longitudinal axis of the primary catheter.

15. The vascular access system of claim 1, wherein a flow path through the secondary lumen to the primary catheter is open when the mid-line lumen is in the retracted position and closed when the mid-line lumen is in the extended position.

16. The vascular access system of claim 15, wherein the primary lumen and the secondary lumen are in fluid communication via a joining connector, and wherein the joining connector comprises a seal configured to close the flow path through the secondary lumen to the primary catheter when the mid-line lumen is in the extended position.

17. The vascular access system of claim 16, wherein the mid-line lumen comprises a first portion extending from the distal end of the mid-line lumen to a position intermediate the distal end of the mid-line lumen and a proximal end of the mid-line lumen and a second portion extending from the proximal end of the mid-line lumen to the first portion of the mid-line lumen, wherein the first portion comprises a first material and the second portion comprises a second material, and wherein the second material is stiffer than the first material.

18. The vascular access system of claim 16, wherein the mid-line lumen comprises a tapered portion, and wherein the tapered portion of the mid-line lumen engages the seal when the mid-line lumen is in the extended position.

19. The vascular access system of claim 15, wherein the primary lumen and the secondary lumen are in fluid communication via a joining connector, and wherein the mid-line lumen comprises a seal configured to engage a portion of the joining connector to close the flow path through the secondary lumen to the primary catheter when the mid-line lumen is in the extended position.

20. The vascular access system of claim 1, wherein the primary lumen and the secondary lumen are in fluid communication via a joining connector, wherein a seal actuator is at least partially received within the joining connector, and wherein movement of the seal actuator is configured to close a flow path through the secondary lumen to the primary catheter.

21. The vascular access system of claim 1, wherein the distal end of the mid-line lumen comprises a slit having an open position allowing fluid to flow out of or into the mid-line lumen and a closed position.

22. The vascular access system of claim 1, wherein a distal end of the primary catheter comprises an opening defined by a sidewall of the primary catheter.

23. A method of using the vascular access system of claim 1, the method comprising:
attaching a blood collection device to the secondary lumen port;
moving the mid-line lumen to the extended position; and
removing the blood collection device from the secondary lumen port.

24. A method of using the vascular access system of claim 1, the method comprising:
attaching a fluid source to the secondary lumen port;
moving the mid-line lumen to the extended position; and
delivering fluid from the fluid source through the mid-line lumen.
